(19) United States
(12) Patent Application Publication
(10) Pub. No.: US 2008/0269367 A1
(43) Pub. Date: Oct. 30, 2008

(54) Prepolymer containing a Liquid Hardness Agent For Open Cell Foams

(76) Inventors: Paul L. Neill, Grayslake, IL (US); Warren A. Kaplan, Graylake, IL (US); Douglas Kip Hillshafer, Western Springs, IL (US)

Correspondence Address: MCANDREWS HELD & MALLOY, LTD, 500 WEST MADISON STREET, SUITE 3400, CHICAGO, IL, 60661, US

(21) Appl. No.: 12/089,339

(22) PCT Filed: Oct. 17, 2006
(86) PCT No.: PCT/US06/40528

§ 371 Date: Jun. 25, 2008

Related U.S. Application Data
(60) Provisional application No. 60/727,653, filed on Oct. 18, 2005.

(51) Int. Cl. C08G 18/40; C07C 265/00; C08G 18/10

(52) U.S. Cl. 521/137; 252/183.11

(57) ABSTRACT

The present technology provides at least one prepolymer comprising a polyisocyanate and a ring-containing isocyanate reactive component as a load-bearing capacity improvement agent in flexible polyurethane foam manufacture. The ring-containing component eliminates and/or reduces the need for a copolymer polyol containing suspended solids in the manufacture of flexible foam products. This can reduce production costs, reactivity variations, filter plugging, color variations, foam shrinkage/tightness, foam irregularity, and foam malodor while maintaining an adequate load-bearing capacity.

### BACKGROUND OF THE INVENTION

The present technology relates to open cell polyurethane foams made from polyisocyanates and polyols. In at least one aspect, the present technology provides a prepolymer for use in making a blend foam intermediate composition, a foam composition, flexible material and articles made from the foam, and methods of making the same, that contain at least one ring-containing isocyanate-reactive component capable of aiding in the control of the load-bearing capacity of flexible polyurethane foams.

### PRIOR ART FOAM FORMULATION

Flexible polyurethane foams are chemically and physically complex synthetic materials formed by the reaction of polyisocyanate compounds with polyol resin compounds in the presence of a number of other formulation ingredients. Flexible polyurethane foams are typically produced using either slabstock foam manufacturing processes or molded flexible foam manufacturing processes. One example of a formulation which can be utilized for flexible foam manufacturing processes is set forth below:

| Component | Parts |
|---|---|
| ARCOL F-3222 Polyol | 70 |
| HX 100 Copolymer Polyol | 30 |
| NIAX L-620, Surfactant | 1.2 |
| T-Cat 100, Stannous Octoate Catalyst | 1.4 |
| DABCO 33-LV, amine catalyst | 0.35 |
| NIAX A-1, amine catalyst | 0.08 |
| Water | 4.2 |
| Toluene Diisocyanate Index | 80-120 |

In this particular formulation, HYPERLITE E-848 Polyol serves as a base polyol component and HYPERLITE E-849 is a copolymer polyol component, each commercially available from Bayer Corporation of Leverkusen, Germany. The base polyol is typically the major component of a foam formulation and is generally chosen with regard to the bulk properties desired in the final foam. The copolymer polyol is a dispersion of microscopic, discontinuous, solid polymer particles in a continuous polyol matrix. The copolymer polyol functions as a load-bearing adjusting additive.

Surfactants such as NIAX Y-10184 aid in reducing the surface and interfacial tensions between various components of the foam producing formulations. NIAX Y-10184 is a silicone-based surfactant commercially available from GE-Silicones.

Diamine amine functions as a stabilizing crosslinker, and DABCO 33-LV and NIAX A-1 amine catalysts that aid in the polymerization and curing of the foam intermediate composition into a resultant foam product. DABCO 33-LV is a 33 weight % solution of triethylene diamine in dipropylene glycol and is commercially available from Air Products and Chemicals, Incorporated, Allentown, Pa. NIAX A-1 is a 70 weight % solution of bis(dimethylaminoethyl) ether in dipropylene glycol and is commercially available from Crompton OSI Specialties, Incorporated, Middlebury, Conn.

Toluene diisocyanate (TDI) is used to react with the active hydrogen ingredients in the listing to produce a high molecular weight polymer. TDI also reacts with the water to generate carbon dioxide which is used as a blowing agent in the reaction mixture to form the cells of the foam.

### Flexible Slabstock Foam

Commercial scale production of flexible slabstock foams began in North America in about 1954 and was based on the use of aliphatic polyester-type polyols (often made into prepolymers prior to the foaming event). These early foams proved unable to withstand many in-use temperature and humidity conditions and often failed by decomposition under these conditions. Improved performance was obtained with the introduction of high-purity polyether-type polyols in about 1957. Foams based on these polyols were less affected by hydrolysis and thus more durable.

A slabstock/one-shot type foam is typically produced in either a high pressure or low pressure machine having a continuous mixer. Such continuous mixing machines can, generally, produce 100 pounds or more per minute of slabstock foam.

In general, the production of slabstock foam involves the metering of foam ingredients from separate feed lines (i.e., streams) via a mixing head having a pin mixer or high shear mixer. Typically, slabstock foam is made from a polyether polyol; a polyisocyanate such as toluene diisocyanate (TDI); an amine catalyst and a tin catalyst. The polyether polyol often comprises mostly secondary OH groups.

Further information regarding the production of slabstock foam can be found in the following references: Frisch, K. C. and Saunders, J. H., Polyurethanes: Chemistry and Technology Part II, High Polymers, vol. XVI, part II, pp. 85-191 (1983); Sandridge, R. L., et al., Effect of Catalyst Concentrations on One-shot Polyether Flexible Urethane Foams, American Chemical Society, Division of Organic Coating Plastics Chemistry, Preprints (1961), 21 (no. 2), pp. 68-78; and U.S. Pat. Nos. 3,194,773 to Hostettler and 3,546,145 to Granger, et al. Such references are illustrative of prior art slabstock foam processing, but are not intended to be an exhaustive list.

### Molded Flexible Foams

The primary characteristic of a foam cushion that is adjusted to meet a particular functional requirement is the load-bearing capacity. The load-bearing capacity of flexible polyurethane foam is its ability to receive and support a given weight at a particular deflection.

Load-bearing capacity in flexible foams, and methods to enhance it, are subjects that have been continuously studied since the foam cushioning industry began (with the installation of natural rubber latex foam seat cushions in London buses in 1932). Methods to improve load-bearing in flexible polyurethane foams have been studied since their commercial introduction in 1954. The following is a list of some of the major known contributing factors to the load-bearing capacity of a flexible polyurethane foam: density, water level, cell size, cell openness, packing, fillers, use of expandable beads, copolymer polyol levels, polyisocyanate type and index, use of cross-linkers, polyol functionality, use of chain extenders, polymer morphology adjustments, and use of specific catalysts.

Of these known methods to adjust the load-bearing capacity of flexible polyurethane foam, the most commonly used method is the inclusion of copolymer polyols in the reacting foam mixture. As mentioned above, copolymer polyols are dispersions of microscopic, discontinuous, solid polymer particles in a continuous polyol matrix. The first commercial copolymer polyols appeared in 1966 and were based on the use of acrylonitrile as the sole monomer. These products were instrumental in the commercial acceptance of molded high-resiliency (HR) foams for use in automobile seating. By the early 1970's, copolymer polyols had become the preferred method for increasing foam load-bearing capacity in polyurethane foams. A wide range of foam hardness could be obtained simply by using more or less of the product. The solid particles behaved as a classical filler, but were available in an easy to use liquid form. Over the years, deficiencies in the 100% acrylonitrile products led to the development of styrene-acrylonitrile (SAN) copolymer polyols. The following patents are some examples of the many patents and patent applications in this area: U.S. Pat. Nos. 3,304,273 to Stamberger; 3,523,093 to Stamberger; 3,931,092 to Ramlow et al.; 3,953,393 to Ramlow et al.; 4,104,236 to Simroth; 4,186,271 to Preston et al.; 4,242,476 to Shah et al.; 4,390,645 to Hoffman et al.; 4,454,255 to Ramlow et al.; 4,495,341 to Stamberger; 4,521,546 to O'Connor et al.; 4,539,339 to Cuscurida et al.; 4,539,378 to Cuscurida et al.; 4,745,153 to Hoffman; 4,931,483 to Matsuoka et al.; 5,171,759 to Hager; 5,741,847 to Willkomm et al.; 6,455,185 to Bircann et al.; Re. 28,715 to Godfrey; Re. 29,118 to Woodward; GB 2070628 to Mellado et al.; GB 2179356 to Mellado et al.; GB 2309700 to Breukel et al.; WO 94/20558 to Vandichel et al.; WO 97/15606 to Thomas et al.; WO 99/40144 to Eleveld et al.; WO 00/00531 to Fogg; and WO 01/09242 to Hiroyuki et al.

Although commercially successful, the SAN type copolymer polyols are not without deficiencies.

Manufacturers of copolymer polyols normally ship product at plus or minus 2 weight percent (i.e., wt % or weight %) of the target percent solids specification. This means, for example, that shipments of a nominal 40% solids copolymer polyol could arrive containing anywhere from 38% to 42% solids.

Variations in the weight percent solids in the delivered copolymer polyol product and variations in the average particle size contribute to variations in the load-bearing capacity observed in the final produced foam. Foam manufacturers normally have multiple foam recipes that they use daily in each of their production plans to make a wide variety of foams as desired. These recipes are assigned around the copolymer polyol product having some nominal weight percent solids level and are not typically adjusted for the actual solids level of each incoming lot of product. Thus, variation in the actual weight percent of solids and the resultant load-bearing capacity is inherent in the current start and stop, semi-continuous processes used to make molded flexible foam.

Further, as the actual level of solids varies in the copolymer polyol product so will the viscosity of the neat copolymer polyol. Variations in the concentration of particle stabilizer also contribute to viscosity variations in the neat copolymer polyol. It is not uncommon for viscosities of individual shipments of copolymer polyol to vary plus or minus 1000 mPa·s at 25° C. around the product's target specification.

Moreover, when the copolymer polyol is formulated into a masterbatch with the other ingredients, the viscosity of that masterbatch increases in direct relationship to the amount of copolymer polyol used. In some cases, the resultant masterbatch viscosity can be so high as to challenge the pumping and metering capabilities of the foam making equipment.

A common industrial problem with using copolymer polyols is the plugging of filters located at several key points in a foam production process. The filters can be used to catch solid contaminants arising from normal shipping and handling operations so that processing upsets and down time do not affect plant operations. Even though copolymer polyols are typically specified as having average particle sizes in the range of 1 micron or smaller, it is common for any given shipment of product to plug 100 micron and even larger sized filters.

Further, variations in the reactivity of known copolymer polyols arise from normal lot-to-lot variability in the polyol which is used to carry the suspended styrene/acrylonitrile particles.

Known copolymer polyols present additional problems in that the color of a neat copolymer polyol product can vary and this in turn can vary the color of the foam produced. Color variations arise from intended or unintended changes in the ratio of styrene to acrylonitrile monomers. The usual color of a copolymer polyol product is white, but the color of the resulting foam can have a yellowish hue as the ratio of acrylonitrile to styrene is increased. Further problems can result from the overheating of a copolymer polyol at the foam manufacturing plant during off-loading of the copolymer polyol in the winter, resulting in a more yellow colored product and final foam.

Still further, the odor of the copolymer polyol often transfers into the foam production plant and into the foams produced therein. This presents important quality issues. In the copolymer polyol product, odors can arise from the presence of residual styrene or acrylonitrile monomers as well as from the myriad of free radical fragments formed during polymerization.

A number of preferred alternative types of copolymer polyols have been studied and reported, such as, for example, polyurea copolymer polyols (see U.S. Pat. Nos. 3,325,421 to Muller; 4,089,835 to König et al.; 4,093,569 to Reischl et al.; 4,107,102 to Dahm et al.; 4,296,213 to Cuscurida et al.; 4,523,025 to Cuscurida et al.; and 4,761,434 to Dietrich et al.); polyisocyanate polyaddition copolymer polyols (see U.S. Pat. Nos. 3,360,495 to Muller et al.; 4,260,530 to Reischl et al.; 4,374,209 to Rowlands et al.; 4,438,252 to Carroll et al.; 4,452,923 to Carroll et al.; 4,497,913 to Raes et al.; 4,525,488 to Cuscurida et al.; 4,554,306 to Carroll; 4,595,709 to Reischl; 4,785,026 to Yeakey et al.; 5,068,280 to Pal et al.; 5,179,131 to Wujcik et al.; and 5,292,778 to Van Veen et al. and GB 2 102 824 A to Allport); epoxy dispersion copolymer polyols (see U.S. Pat. Nos. 4,305,861 to Marx et al.; 4,789,690 to Milovanovic-Lerik et al.; and 5,244,932 to van der Wal); and miscellaneous other copolymer polyols (see U.S. Pat. Nos. 4,323,657 to Mazanek et al.; 4,326,043 to Narayan et al.; 4,435,527 to Cuscurida; 4,435,537 to Horn et al.; 4,452,922 to Speranza et al.; 4,521,581 to Dominquez et al.; 5,594,072 to Handlin, Jr. et al.; WO 01/88005 to van der Wal et al.; and WO 02/10247 to Van Heumen et al.).

Other alternative approaches include, for example, those reported in U.S. Pat. Nos. 3,454,530 to Case et al.; 3,957,753 to Hostettler et al.; 4,237,240 to Jarre et al.; 4,374,935 to Decker et al.; 4,469,823 to Yeakey et al.; 4,524,157 to Stamberger; 4,568,717 to Speranza et al.; 5,003,027 to Nodelman; and 5,606,005 to Oshita et al.

Notwithstanding its shortcomings, however, until now the use of SAN type copolymer polyols has been the commercially preferred approach for increasing the load-bearing capacity of flexible polyurethane foam.

### BRIEF SUMMARY OF THE INVENTION

It has now been discovered that one or more embodiments of the present technology overcome many of the shortcomings of known foam formulations containing copolymer polyols, either in whole or in part, while achieving adequate load-bearing capacities for various foam products.

It is therefore an object of the present technology to provide one or more embodiments of polyurethane foams that eliminate or reduce one or more of the shortcomings of the prior art.

It is a further object of the present technology to provide at least one embodiment comprising a ring-containing isocyanate-reactive component or ring-containing isocyanate reactive component blend that has reduced levels of suspended solids, such as the SAN particles typically found in some known copolymer polyols.

Another object of the present technology is to provide one or more embodiments that replace the copolymer polyol in part or entirely with another load-bearing additive.

It is yet another object of the present technology to provide one or more embodiments comprising an open cell polyurethane foam derived from a prepolymer comprising polyisocyanate and a ring-containing isocyanate reactive component or ring-containing isocyanate reactive component blend that reduces or eliminates at least some of the shortcomings associated with copolymer polyols including, but not limited to variations in weight percent solids and average particle size; variations in viscosity of the neat product and increased viscosity of the foam formulation masterbatch (due to the inherent high viscosity of copolymer polyols); filter plugging; reactivity variations; color variations; foam slhinkage/tightness; foam irregularity; and/or foam malodor.

Another object of the present technology is to provide one or more embodiments that prepare a prepolymer composition comprising a polyisocyanate and a ring-containing isocyanate reactive component for use in the preparation of a flexible polyurethane foam. Use of such a prepolymer allows load-bearing characteristics to be maintained without significant degradation of other flexible foam properties, and allows a reduction in the amount of copolymer polyol previously required to maintain such characteristics. Further, such a prepolymer provides processing advantages, such as a reduction in the amount of unmodified isocyanate added to the polyether polyol (i.e., the “B” side) to produce the foam, which results in a lower reaction exotherm and improved foam consistency compared to prior art foams prepared with only copolymer polyols to provide the load-bearing characteristics.

Various embodiments of the present technology disclosed herein are intended to address at least in part one or more of the foregoing objects. It should be understood that not every embodiment of the present technology addresses each of the foregoing objects, and that the embodiments disclosed herein should not be limited to the foregoing objects.

Several terms used below are specifically defined as follows.

All parts and percentages herein are by weight unless otherwise indicated or apparent.

A “ring,” as used in the term “ring-containing component polyol” or otherwise, e.g. ring-containing polyamine, ring-containing polyamide, ring-containing polyepoxide, ring-containing polycarboxylic acid, ring-containing polyanhydride, or ring-containing thiol, is broadly defined here to include aromatic monocyclic or polycyclic rings, aliphatic monocyclic or polycyclic rings, and includes carboxylic or heterocyclic rings of any of these types. The ring-containing component optionally can have an isocyanate reactive functionality between 1.7 and 3.5.

By “ring structure content” is meant the percentage by weight of atoms that are part of a ring structure within the theoretical prepolymer molecule.

An “aromatic polyol” is broadly defined here to include aromatic monocyclic or polycyclic rings, and includes carboxylic or heterocyclic rings of either of these types. The aromatic polyol optionally can have an isocyanate reactive functionality between 1.7 and 3.5.

Any reference to a material having a certain characteristic refers either to a single material of a mixture or blend of materials having, in aggregate, that characteristic, unless the context clearly indicates otherwise. For example, unless the context indicates otherwise, a “polyol” refers to a polyol or a polyol blend.

One aspect of the present technology is a prepolymer prepared from a polyisocyanate and at least 35% by weight of the prepolymer of a ring-containing isocyanate reactive component having load-bearing characteristics. The prepolymer can be further mixed with an unmodified polyisocyanate to form the “A” side of a flexible foam formulation.

An additional aspect of the present technology is a flexible foam prepared from (a) a prepolymer comprising a polyisocyanate and at least 35% by weight of the prepolymer of a ring-containing isocyanate reactive component and (b) a polyether polyol. The prepolymer can be further mixed with an unmodified polyisocyanate to form the “A” side of a flexible foam formulation prior to reacting with the polyether polyol (“B” side) to form the flexible foam.

Still another aspect of the present technology is a flexible foam made by reacting ingredients including a polyether polyol, and a prepolymer comprising a polyisocyanate and at least 35% by weight of the prepolymer of a ring-containing isocyanate reactive component, in the presence of a blowing agent, the foam having a density of from 6 to 240 kg/m³ and requiring a 65% indentation deflection of from 9 to 15 Newtons per 323 square cm. per kg/m³.

Additionally, another aspect of the present technology is a process of making a foam article which includes a flexible foam comprising admixing a polyether polyol, a blowing agent, a prepolymer comprising a polyisocyanate and at least 35% by weight of the prepolymer of a ring-containing isocyanate reactive component having load-bearing characteristics, a surfactant, and a catalyst.

One optional feature of each aspect of the present technology is that the ring-containing polyol can be substantially free of aromatic diamines.

An additional aspect of the present technology is a flexible foam having a density of 16 to 144 kg/m³, optionally 26 to 45 kg/m³, made by reacting a prepolymer comprising a ring-containing isocyanate reactive component and a polyisocyanate, with a polyether polyol in the presence of a blowing agent.

The flexible foam of any aspect of the present technology optionally includes an amount of the ring-containing component polyol effective to increase the 65% indentation force deflection (IFD) of the foam.

Yet another aspect of the present technology is a flexible polyurethane foam made from the prepolymer of any aspect of the present technology.

Another optional feature of the foam of any aspect of the present technology is a 65% indentation force deflection guide factor of from 5 Newtons to 20 Newtons, optionally from 7.5 to 20 Newtons, optionally from 4 to 19 Newtons, optionally 7 to 15 Newtons optionally from 7 to 18 Newtons, optionally from 9 to 15 Newtons, optionally from 9 to 18 Newtons, optionally from 11.5 to 20 Newtons, or any other combination of these lower and upper limits, per 323 sq. cm. per kg/m³. The “indentation force deflection guide factor” is defined as the ratio of an indentation value, such as the 65% or 25% IFD, to the density of the foam. This term is useful in determining the relative firmness of foams having different densities.

The foam of any aspect of the present technology optionally has a density of from 6 to 240 kg/m³, optionally from 8 to 160 kg/m³, optionally from 24 to 64 kg/m³.

Yet an additional aspect of the present technology is a flexible foam pad having a density of 16 to 144 kg/m³ made by reacting the prepolymer of any aspect of the present technology with a polyether polyol in the presence of a blowing agent to form a foam intermediate composition, and, fabricating a foam pad from the foam intermediate composition. Optionally, the pad can have a density of 26 to 45 kg/m³. Optionally, the pad can include an amount of the ring-containing isocyanate reactive component effective to increase the 65% IFD of the foam.

### DETAILED DESCRIPTION OF THE INVENTION

Various aspects of the present technology can be carried out by providing, making, or using a prepolymer comprising a ring-containing, load-bearing isocyanate reactive component and a polyisocyanate, and reacting the prepolymer with a base polyol comprising a polyether polyol to prepare flexible foams having excellent load-bearing properties. The ingredients of such a prepolymer, and how it is made and used are further described below.

### “B” Side Components Description

### Base Polyol

High-resiliency molded polyurethane foams are typically prepared using polyether polyols having equivalent weights between about 1000 and 2500 g/equivalent and typically having 5 to 25% by weight end capping with ethylene oxide to provide primary hydroxyl contents ranging from about 65% to 90%. The functionality of these polyols is typically greater than about 2.0, and more preferably above about 2.4. Propylene oxide is preferred as the main comonomer in these types of polyether polyols.

Slabstock polyurethane flexible foams are typically prepared using polyether polyols having an equivalent weight between about 500 and 1600 g/equivalent. These polyols are generally terminated with mainly secondary hydroxyl groups, but can also be end capped with ethylene oxide to increase the primary hydroxy group content. Ethylene oxide can also be randomly copolymerized with other alkylene oxides in these polyols, or block copolymerized to provide increased surfactancy and hydrophilicity. Propylene oxide is preferred as the main comonomer in these types of polyether polyols. The functionality of these polyols is typically greater than about 1.8, and more preferably greater than about 2.2.

An example of a commercially available base polyol for flexible molded foam is HYPERLITE E-848 from Bayer Corporation. HYPERLITE E-848 is an approximately 1800 equivalent weight (approximately 31.5 hydroxyl number) polyether polyol made by adding propylene oxide to an initiator compound to build an intermediate molecular weight and then capping with ethylene oxide to give a final product.

The equivalent weight values noted herein for the base polyol components which can be utilized indicate the mass of polyol per reactive hydroxyl group of the component polyols, and such values are typically expressed in units of g/equivalent.

The OH values noted herein for the base polyol components that can be utilized indicate the number of hydroxyl groups available for reaction in the described polyol, and such values are typically expressed in units of milligrams of KOH per gram of sample.

### Blowing Agents

To prepare foam, water is most preferred for use as the blowing agent. However, any other way known to prepare polyisocyanate-based foams can be employed in addition to or instead of water. For example, the foam can be blown by using reduced or variable pressure, an inert gas such as nitrogen, air, carbon dioxide, argon, or other conventional blowing agents. Some examples of other conventional blowing agents are chlorofluorocarbons, hydrofluorocarbons, hydrocarbons, hydrochlorocarbons, fluorocarbons, other classes of compounds having boiling points between about −20 and 100° C., and reactive blowing agents, i.e., agents which react with any of the ingredients, or decompose with heat in the reacting mixture, to liberate a gas which causes the mixture to foam.

### Catalysts

The catalysts normally used to manufacture flexible foams are suitable for preparing the contemplated flexible foams. Included, for example, are organometallic compounds such as tin (II) salts of organic carboxylic acids and the dialkyl tin (IV) salts of organic carboxylic acids. These compounds can be used alone or preferably in combination with strongly basic amine compounds; tris (N,N-dialkyl aminoalkyl)-s-hexahydrotriazines; tetraalkyl ammonium hydroxides; alkali hydroxides; alkali alkoxides; alkali salts of long-chain fatty acids, optionally having side-positioned hydroxyl groups; alkali salts of N-(2-hydroxy-5-nonylphenol) methyl-methyl glycinate; and mixtures thereof. The preferred amine catalysts are tertiary amine compounds, while the preferred organometallic catalysts are based on tin. More preferred catalysts include, but are not limited to, 33 weight % solution of urethylene diamine in dipropylene glycol, available under the trademark DABCO 33-LV from Air Products and Chemicals, Incorporated, a 70 weight % solution of bis(dimethylaminoethyl) ether in dipropylene glycol, available under the trademark NIAX A-1 from Crompton-OSI Specialties, and an octoate salt such as potassium octoate. Additional catalysts suitable for use with the present technology include, for example, trimerization catalysts. Trimerization catalysts are contemplated to aid the cure of the foam and to make the resulting foam harder. Some examples of trimerization catalysts are listed as “additional catalysts/additives” in Examples 3, 6, 8, 10, 13, 15-17, 19, 25-27, 29-37, and 40-42 of Table 4 in U.S. Patent Application Publication No. 20040259967, to Neill et al., the content of which is hereby incorporated by reference.

### Buffers

Another contemplated foam formulation ingredient is a buffer. Exemplary buffers contemplated herein include, but are not limited to, alkali carbonate salts, alkali bicarbonate salts, and mixtures thereof. Some specific buffers contemplated here include, but are not limited to, sodium carbonate, sodium bicarbonate, potassium carbonate, potassium bicarbonate, and mixtures thereof, either added as separate ingredients or formed in situ.

### Surfactants

Typical surfactants which can be used include, but are not limited to, nonionic surfactants such as MAKON® surfactants sold by Stepan Company; silicone-based surfactants such as NIAX Y-10184 and NIAX L-620 surfactants which are available from GE Silicones; and TEGOSTAB B 4690 available from Degussa-Goldschmidt Chemical.

### Cross-Linkers

Examples of cross-linkers that can be used include, but are not limited to, glycerin, trimethylol propane, diethanol amine, triethanol amine, and four or more functional polyols intended primarily for making rigid polyurethane foam.

### Copolymer Polyols

An example of a copolymer polyol component suitable for use as an optional agent is HYPERLITE E-849, commercially available from Bayer Corporation. This particular copolymer polyol is a nominal 40 weight % styrene/acrylonitrile solids containing 18.4 OH number (3049 equivalent weight) polyol that is designed for use as a hardness adjusting agent in making molded polyurethane foams. Another example of a copolymer polyol that can be incorporated into flexible foams of the present technology includes HS100, a nominal 43 weight % styrene/acrylonitrile solids containing copolymerized polyol with an OH number of 28. Additional copolymer polyols that can be optionally incorporated into flexible foams of the present technology include those described within the background of the Invention section set forth above.

### Dendritic Macromolecules

An optional additive in the present compositions, contemplated for use as an auxiliary load-bearing improvement agent, is a dendritic macromolecule as discussed in PCT application WO 02/10247 A1 to Van Heuman et al., with reference to U.S. Provisional Application 60/221,512. A more extensive discussion of what is a dendritic macromolecule can be found in U.S. Pat. No. 6,093,777 to Sorensen et al. All the patents and applications mentioned in this paragraph are hereby incorporated herein by reference to describe these dendritic macromolecules, how to make them, and how to use them in polyurethane foam compositions.

### Inorganic Particulates

In some embodiments, inorganic particulates, such as fire retardant inorganic particulates, can be excluded from the formulations to reduce the potential for settling, separation and clogging of filters. In an alternative embodiment, however, low levels of inorganic particulates can be incorporated, preferably with relatively continuous agitation. Preferably, the level of inorganic particulates is less than about 10% by weight; more preferably less than about 5% by weight and most preferably below about 1% by weight.

### “A” Side Components Description

### Prepolymer Component Description:

Prepolymers comprising polyisocyanates and ring-containing isocyanate reactive components can be prepared by reacting one or more ring-containing isocyanate reactive components described herein with one or more polyisocyanates described herein. The ring-containing isocyanate reactive components include ring-containing polyols, ring-containing polyamines, ring-containing polyepoxides, ring-containing polyamides, ring-containing polycarboxylic acids, ring-containing polyanhydrides, and ring-containing polythiols.

Catalysts can be used in the preparation of the prepolymers; however, it is generally desirable to avoid the use of catalysts, in order to provide a prepolymer that does not undergo further polymerization during storage. Catalysts suitable for the preparation of the prepolymer component include tin compounds such as, for example, alkyl tin carboxylates and tin carboxylates, transition metal compounds and tertiary amines.

The prepolymers can be prepared by simply reacting one or more of the isocyanate components described herein with one or more of the ring-containing isocyanate reactive components described herein in a suitable reaction vessel, in a dry atmosphere, (i.e., under a nitrogen blanket). The components can be added together at ambient temperature and then heated with stirring, or the ring-containing isocyanate reactive component or component mixture can be heated and then added to the isocyanate component with stirring. The mixture is usually heated to between about 40 and 120° C. for a period of time generally ranging between 15 minutes and 12 hours until the desired NCO content is achieved, after which the reaction product is discharged.

The presence of water in the isocyanate reactive component is generally undesirable. However, low levels of water can be included along with the ring-containing isocyanate reactive component, as long as the water is accounted for in the stoichiometry and as long as the water does not create overflow or unacceptable pressurization of the reaction vessel and, preferably, as long as the final viscosity of the resulting prepolymer remains below about 100,000 cps at 25° C.

The quantity of ring-containing isocyanate reactive component that is reacted with the isocyanate component is at least about 35% by weight relative to the final weight of the prepolymer, preferably at least about 40% by weight relative to the final weight of the prepolymer. The amount of the isocyanate component in the prepolymer is preferably no greater than about 65% by weight of the prepolymer. The stoichiometry can be selected such that sufficient equivalents of isocyanate remain after reaction with the ring-containing isocyanate reactive component so that the prepolymer will be isocyanate terminated. Alternatively, the stoichiometry can be selected such that sufficient equivalents of hydroxyl remain after reaction with the isocyanate component so that the prepolymer will be hydroxyl terminated. In either case, the final viscosity of the prepolymer should be such that it can be readily processed to form a flexible foam, preferably less than about 100,000 centipoise at 25° C.

If the prepolymer is isocyanate-terminated, it can be further blended with an unmodified isocyanate component to form the A side prior to reaction with a polyether polyol (i.e. B side) to form the flexible foam. In such an embodiment, the A side can comprise from about 20% to about 50% of the prepolymer and from about 80% to about 50% of the isocyanate, preferably about 30% of the prepolymer and 70% of the isocyanate.

Without wishing to be bound by a particular theory, it is believed that by reacting the ring-containing isocyanate reactive component with the isocyanate to form at least one prepolymer, better incorporation of the ring-containing component into the flexible foam can be achieved than if the ring-containing component were not formed into a prepolymer. The incorporation of the ring-containing component into the flexible foam provides enhanced load-bearing characteristics in the flexible foam without significant degradation of other flexible foam properties. Moreover, use of the prepolymer provides an ability to maintain load-bearing and other physical properties, including, but not limited to, tensile strength, tear strength, and elongation, while reducing the copolymer polyol level within the flexible foam formulation. As is well known in the art, reduction in copolymer polyol level is expected to lead to significant enhancement of foam fatigue properties. In general, the load-bearing properties are achieved when the ring structure content of the prepolymer is at least about 25% by weight of the prepolymer, preferably at least about 30% by weight of the prepolymer.

Again, without wishing to be bound by a particular theory, it is believed that the use of the ring-containing polyol prepolymer also provides processing advantages, such as a reduction in the amount of isocyanate required during foam formation and a lower reaction exotherm during foaming. By controlling the heat generated during foaming through use of the prepolymer, the properties of the foam, such as density and load-bearing characteristics, can be better controlled, resulting in flexible foams having more uniform properties throughout the resulting foam. For example, in at least one embodiment of the present technology, the amount of isocyanate added during foam forming can be reduced by about 10 to about 30% compared to prior art foam processes that do not utilize a prepolymer.

In an alternative embodiment of at least one prepolymer of the present technology, it is contemplated that the prepolymer can be prepared by reacting one or more ring-containing polyisocyanates described herein with an acylic component, such as, for example a low molecular weight diamine or amide. In such an alternative embodiment, the polyisocyanate serves as the ring-containing load-bearing component for the prepolymer.

### Ring-Containing Component Polyol

Ring-containing component polyols for use in forming prepolymers of the present technology cancan include, but are not limited to, aromatic polyester polyols; heterocyclic polyols; spiro ring-containing component polyols; fused ring-containing component polyols; ring-containing component polyether polyols such as, for example, alkoxylated sucrose polyols; ring-containing component polyester polyols; alkoxylated ring-containing component aromatic and aliphatic diamines; alkoxylated phenol/formaldehyde resins; alkoxylated bis- or poly-phenols; alkoxylated dihydroxy benzenes and derivatives thereof; Mannich-type polyols; 1,4-cyclohexane dimethanol; dimethylol cyclopentadiene; alkoxylated piperazine; alkoxylated di- or poly-hydroxy naphthalenes; dihydroxy cyclohexane isomers; halogenated ring-containing component polyols; combinations thereof and derivatives thereof.

Many ring-containing component polyols of the present technology are prepared as a result of alkoxylation of a ring-containing component initiator. This alkoxylation process occurs during the ring opening reaction or polymerization of an alkylene oxide or mixture of alkylene oxides or sequential addition of different alkylene oxides to a suitable ring-containing component initiator having active hydrogens in its structure. These reactions are usually carried out in the presence of either an acid or base catalyst. Alkylene oxides suitable for use in alkoxylating ring-containing component initiators include ethylene oxide, propylene oxide, butylene oxide, propylene carbonate, ethylene carbonate, styrene oxide, and epichlorohydrin. Alkoxylation frequently affords a number of benefits over using an active hydrogen-containing, ring-containing component initiator directly in the practice of the present technology. The toxicity of certain ring-containing component aliphatic and aromatic amines may reduce their use in certain flexible foam applications. Alkoxylation of such ring-containing component amines may reduce their toxicity to permit their use in foam applications.

Other ring-containing component polyols are identified, for example, in U.S. Pat. Nos. 6,359,022 to Hickey et al.; 5,922,779 to Hickey; 4,722,803 to Magnus et al.; 4,615,822 to Magnus; 4,608,432 to Magnus et al.; 4,526,908 to Magnus et al.; and 4,521,611 to Magnus; all issued to the present assignee and incorporated by reference for their description of ring-containing component polyols.

It is preferred that the average functionality of the ring-containing component polyol be high enough to avoid excessive chain termination during the polymerization process and hence allow growth in the overall molecular weight of the polymer. Thus, it is preferred that the average functionality of the ring-containing component polyol or ring-containing component polyol blend be greater than about 1.4, more preferred that the average functionality be greater than about 1.6, and most preferred that the average functionality be greater than about 1.7.

It is preferable that the average functionality of the ring-containing component polyol be low enough to avoid reductions in tensile strength, elongation and tear properties of the foams that are formed. Thus, it is preferred that the average functionality of the ring-containing component polyol or ring-containing component polyol blend be less than about 3.5, more preferably less than about 2.9 and most preferably below about 2.6. While many of the contemplated ring-containing component polyols possess a functionality outside of the range stated above, such polyols can be used in a ring-containing component polyol blend that would result in the polyol blend having an average functionality in the range stated above.

Ring-containing component polyols preferably have a hydroxyl value (OHV) of less than 250, more preferably in the range of about 50 to about 150.

Ring-containing component aromatic and aliphatic amines which can be alkoxylated to form ring-containing component polyols and ring-containing component polyol blends include those having active hydrogens to allow them to be alkoxylated. Examples of the ring-containing component aliphatic amine alkoxylation initiators include, but should not be limited to the following amines, their substituted derivatives and isomers thereof; cyclohexyl amine; cyclopentyl amine; cyclohexane diamine; isophorone-diamine; piperazine; methylene bis(cyclohexylamine); methyl cyclohexane diamine; dimethyl cyclohexane diamine; and amino-1-alkyl piperidine and blends thereof. Additionally, the ring-containing component aliphatic amines can be heterocyclic in nature.

Examples of ring-containing component aromatic amine alkoxylation initiators include, but should not be limited to the following amines, their substituted derivatives and isomers thereof; methylene dianiline; aniline; toluene diamine; polymeric versions of methylene dianiline; diamino benzene; melamine; 4,4-methylene-bis-(2-chloroaniline); methylol melamines (such as those described in U.S. Pat. No. 4,458,034 to Fracalossi et al.) N,N′-dialkyl methylene dianilines; N,N′-dialkyl phenylene diamines; diethyl toluene diamines; sulfonyl dianiline; isopropylidene dianiline; amino-benzylamines; chloro-anilines; phenylene diamine; benzyl amine; triphenyl methyl amine; and aminopyridine.

Examples of suitable heterocyclic ring-containing component polyols include, but should not be limited to: furan based polyols (such as those described in U.S. Pat. Nos. 4,316,935 to Moss and 4,426,460 to Pentz); alkoxylated melamines (such as those described in U.S. Pat. Nos. 4,656,201 to Marx et al.; 3,812,122 to Lengsfeld; and 5,254,745 to Jähme); alkoxylated sucrose based polyols (such as those described in U.S. Pat. No. 3,153,002 to Wismer et al.); alkoxylated lactose based polyols; alkoxylated fructose based polyols; alkoxylated methyl glucoside polyols (such as those described in U.S. Pat. No. 4,359,573 to Fuzesi et al.); dihydroxy dioxane derivatives; trihydroxy dioxane derivatives; tetrahydroxy dioxane derivatives; polyhydroxy imidazolidone and alkoxylated polyhydroxy imidazolidones (such as those described in U.S. Pat. No. 5,238,971 to Su et al.); and alkoxylated piperazines.

Mannich polyols are well known in the art and are prepared by alkoxylating a condensation product of a phenol or substituted phenol, formaldehyde and a dialkanol amine. The preparation of these polyols is described, for example, in U.S. Pat. Nos. 4,371,629 to Austin; 3,297,597 to Edwards et al.; 4,137,265 to Edwards et al.; 4,383,102 to McDaniel et al.; 6,281,393 to Molina et al.; and 4,883,826 to Marugg et al.; which are hereby incorporated by reference in their entireties. Additionally, although U.S. Pat. No. 4,371,629 B1 to Austin describes blends of Mannich polyols with flexible foam type polyols, this reference uses Mannich polyols in their pure state as crosslinking blend components, meaning that they have a functionality of about 4.0 or more which can cause losses in tensile, tear and elongation properties.

Alkoxylated phenol/formaldehyde resins are another example of suitable ring-containing component polyols. Polyols based on novolac resins or phenol/formaldehyde condensation resins are also well known in the art and are prepared by alkoxylating a condensation product of a phenol with formaldehyde. The preparation of these polyols is described, for example, in U.S. Pat. Nos. 3,497,465 to Kujawa et al.; 3,686,106 to Tideswell et al. and 4,107,229 to Tideswell et al.

Another example of a class of suitable ring-containing component polyols is the group consisting of the alkoxylated bisphenols. Alkoxylated bisphenols can be prepared by reacting the appropriate bisphenol with an alkylene oxide in the presence of an acidic or basic catalyst. Examples of bisphenols suitable for use as initiators in the practice of the present technology include, but should not be limited to: methylene bisphenol; sulfonyl diphenol; isopropylidene bisphenol; isopropylidene bis(dimethyl phenol); hexafluoroisopropylidene diphenol; isopropylidene bis(dibromo phenol); bis(4-hydroxyphenyl)-2,2-dichloroethylene; 4,4′-cyclohexylidene bisphenol; 4,4′-ethylidenebisphenol; 4,4′-(1,3-phenylenediisopropylidene) bisphenol; 4,4′-(1-phenylethylidene)bisphenol; 2,2′-dihydroxybiphenyl; 4,4′-dihydroxybiphenyl; di- or poly-hydroxyl naphthalenes; and mixtures thereof.

Another example of a class of suitable ring-containing component polyols includes the halogenated ring-containing component polyols. Examples of halogenated ring-containing component polyols suitable for the practice of the present technology include, but should not be limited to: glycol diesters of 3,4,5,6-tetrabromo-1,2-benzene dicarboxylic acid or anhydride (the diethylene glycol/propylene glycol mixed ester is sold as PHT-4 Diol by Great Lakes Chemical Corporation, and described in U.S. Pat. No. 4,069,207 to Klein) and polyhydric alcohols derived from hexahalocyclopentadiene as described in U.S. Pat. No. 3,146,220 B1 to Hindersinn et al.

Ring-containing component polyester polyols are the product of esterification of a polyol and a polybasic acid, ester or anhydride or a polybasic acid, ester or anhydride mixture, wherein at least one of the two reactants contains a ring structure. The most common commercially available, ring-containing component polyester polyols are the aromatic polyester polyols, which are well known in the art (see for example, U.S. Pat. Nos. 4,608,432 to Magnus; 4,526,908 to Magnus et al.; 4,529,744 to Wood; 4,595,711 to Wood; 4,521,611 to Magnus; 4,722,803 to Magnus et al.; 4,644,027 to Magnus et al.; 4,644,047 to Wood; 4,644,048 to Magnus et al.; 6,359,022 to Hickey et al.; 5,922,779 to Hickey; 4,758,602 to Trowell; 4,701,477 to Altenberg et al.; 4,346,229 to Derr et al.; 4,604,410 to Altenberg; 5,360,900 to DeLeon et al.; 4,048,104 to Svoboda et al.; 4,485,196 to Speranza et al.; 4,439,549 to Brennan; 4,615,822 to Magnus; 4,753,967 to Londrigan and WO 99/425,508. Each of these patents and the PCT application are incorporated by reference here in their entirety). These aromatic polyester polyols are prepared by forming esters between aromatic di- or poly-basic acids, esters or anhydrides and polyols or glycols.

Examples of aromatic di- and poly-basic acids, esters or anhydrides suitable for use in preparing aromatic polyester polyols include, but are not limited to: phthalic anhydride; dimethyl terephthalate; terephthalic acid; isophthalic acid; 1,8-naphthalic anhydride; 1,8-naphthalic dicarboxylic acid; 1,8-dimethyl naphthalate; dimethyl isophthalate; phthalic acid; dimethyl terephthalate bottoms; phthalic anhydride bottoms; pyromellitic anhydride; mellitic anhydride; mellitic acid; trimellitic anhydride; 3,3′4,4′-benzophenone tetracarboxylic anhydride; 3,3′4,4′-benzophenone tetracarboxylic acid; trimellitic acid; polyethylene terephthalate recycled polymer; polybutylene terephthalate recycled polymer; polyethylene terephthalate virgin polymer; polybutylene terephthalate virgin polymer; mixtures thereof; and the like.

Preferred ring-containing polyols for use with the present technology are the aromatic polyester polyols derived from the reaction product of phthalic anhydride and diethylene glycol. Such polyester polyols should have a hydroxyl value of less than 250, preferably within the range of about 50 to about 150, and a molecular weight of greater than about 449, more preferably within the range of about 748 to about 2244. It will be appreciated by those skilled in the art that the functionality of the polyester polyols, and indeed the functionality of any of the ring-containing isocyanate reactive components, can vary

Accordingly, polyester polyols having a functionality of greater than 2 should have an equivalent weight of greater than about 224, preferably in the range of about 374 to about 1122.

It is also important that such polyester polyols have free (i.e., unreacted) and reacted DEG contents within defined ranges. Whenever a phthalic anhydride/diethylene glycol polyester polyol is made, an oligomer distribution exists-that is dependent upon the molecular weight of the polyol. Within this oligomer distribution, free or unreacted DEG exists. Without wishing to be bound by a particular theory, it is believed that the amount of free DEG present can affect the compression set properties or other fatigue properties of the resulting flexible foam, and that, by minimizing the amount of free DEG present in the oligomer, compression set and other fatigue properties can be optimized. Accordingly, if the phthalic anhydride diethylene glycol polyester polyol has an OHV of about 250, and a molecular weight of about 449 or an equivalent weight of about 224, the polyester polyol has an overall DEG content of about 58% based on the final weight, and a free DEG content of no greater than about 10%. More preferably, the phthalic anhydride/diethylene glycol has an OHV in the range of about 50 to about 150, a molecular weight in the range of about 748 to about 2244, or an equivalent weight in the range of about 374 to about 1122, an overall DEG content in the range of about 47% to about 53%, and a free DEG content of less than 1% to about 4.0%.

Aromatic polyester polyols for use with the present technology can, optionally, be mixed with a monobasic acid. Suitable monobasic acids include, but are not limited to vegetable oils. One preferred vegetable oil is soybean oil.

Examples of aliphatic ring-containing component dibasic acids suitable for use in preparing aliphatic ring-containing component polyester polyols include, but are not limited to: 1,4-dicyclohexane dicarboxylic acid; tetrahydrophthalic acid; tetrahydrophthalic anhydride; 5-norbornene-2,3-dicarboxylic acid and its anhydride; 5-norbomane-2,3-dicarboxylic acid and its anhydride; and 1,4-dimethyl cyclohexane dicarboxylate.

If aliphatic, acyclic or linear mono- di- and poly-basic acids, anhydrides or esters are used in combination with the above described aromatic di- and poly-basic acids, esters or anhydrides, it is preferred that they be present at 20 mole % or less, based on the total amount of di- or poly-basic acid present.

Preferred examples of glycols suitable for use in preparing the ring-containing component polyester polyols can include: glycerine; ethylene glycol; diethylene glycol; triethylene glycol; tetraethylene glycol; propylene glycol; dipropylene glycol; tripropylene glycol; tetrapropylene glycol; trimethylene glycol; 1,1,1-trimethylol ethane; 1,2,3-trimethylolpropane; pentaerythritol; and poly(oxyalkylene) polyols in which each repeating unit contains two to four carbon atoms derived from the condensation of ethylene oxide, propylene oxide, or butylene oxide and mixtures thereof; 2-methyl-1,3-dihydroxy propane; and mixtures thereof to form equivalent weights from about 116 to about 2000 g/equivalent. Optionally aromatic polybasic acids, esters or anhydrides can be directly alkoxylated with ethylene oxide, propylene oxide, butylene oxide or mixtures thereof to provide suitable non-halogenated aromatic polyesters.

Suitable ring-containing component polyester polyols can be prepared from acyclic aliphatic di- or poly-basic acids or blends thereof and ring-containing component polyols. Preferably, aliphatic dicarboxylic acids (or their alkyl esters) having 2 to 12 carbons, more preferably 4 to 8 carbon atoms in the alkylene radical are used. Examples of these dicarboxylic acids (or their alkyl esters) include but should not be limited to: succinic; glutaric; pimelic; undecanoic; dodecanoic; dodecanedioic; subaric; azelaic; sebacic; and most preferably adipic and mixtures thereof. Examples of suitable ring-containing component polyols include, but should not be limited to: heterocycle-containing diols; spiro ring-containing component diols; fused ring-containing component diols; alkoxylated ring-containing component aromatic and aliphatic mono- and poly-amines; alkoxylated phenol/formaldehyde resins; alkoxylated bis- or poly-phenols; alkoxylated dihydroxy benzenes and derivatives thereof; Mannich-type polyols; 1,4-cyclohexane dimethanol; dimethylol cyclopentadiene; alkoxylated piperazine; alkoxylated di- or poly-hydroxy naphthalenes; dihydroxy cyclohexane isomers; halogenated ring-containing component polyols; combinations thereof and derivatives thereof.

Primary, secondary and tertiary aliphatic hydroxyl functionalities are all suitable for the formation of the ring-containing component polyol blends. However, ring-containing component polyols which contain either substantially all primary hydroxyls or which contain mixtures of primary hydroxyls with secondary hydroxyls are preferred.

### Ring-Containing Component Polyol Blend

Additionally contemplated is a ring-containing component polyol blend of any two or more of the ring-containing component polyols discussed above. For example, some of the blends of the present technology include, but are not limited to, ring-containing component polyol blends; non-halogenated ring-containing component polyol blends; non-halogenated aromatic polyester polyol blends; combinations thereof; and derivatives thereof. It is further contemplated that the non-halogenated ring-containing component polyol component of the present technology can be a mixture of the ring-containing component polyols.

Preferably, mixtures of ring-containing component polyols have hydroxyl functionalities of between about 1.7 and 3.5. In one embodiment, ring-containing component polyol blends contain at least two secondary functional hydroxyl groups. In another embodiment, the ring-containing component polyol blends have primary hydroxyl groups. In a further embodiment, the ring-containing component polyol has both primary and secondary OH groups.

As with respect to ring-containing component polyols of the present technology, it is also preferable that the ring-containing component polyol blends and overall blends have reduced levels of suspended solids.

### Other Isocyanate Reactive Ring-Containing Components

In addition to the ring-containing polyols described above, there are other isocyanate reactive ring-containing components that can react with the polyisocyanate component to form the prepolymer of the present technology. Such ring-containing components should have at least two isocyanate-reactive groups and have an equivalent weight of greater than about 224, more preferably an equivalent weight within the range of about 374 to about 1122.

Suitable isocyanate reactive ring-containing components that can be used to form the prepolymer include aromatic or cycloaliphatic primary or secondary polyamines; polyamides; polycarboxylic acids; polyepoxides; polyanhydrides; and polythiols. Examples of polyamines that can be used as the isocyanate reactive ring-containing component include, but are not limited to, 2-4-toluene diamine and 1,4-cyclohexane diamine. Examples of suitable polyamides include, but are not limited to cyclopentyl benzoyl diamide, and the reaction product of phthalic acid or terephthalic acid with a primary amine. Examples of suitable polycarboxylic acids include, but are not limited to 1,4-benzene-dicarboxylic acid and 1,2-benzenedicarboxylic acid. Examples of suitable polyepoxides include, but are not limited to, Bisphenol A diglycidyl ether or 4,4′-isopropylidenedicyclohexyl diglycidyl ether. Examples of suitable polyanhydrides include, but are not limited to, benzophenone-3,3′, 4,4′ tetracarboxylic dianhydride and benzene-1,2,4,5-tetracarboxylic dianhydride. The polythiols that can be used herein are those polythiols that have an active hydrogen that can react with the polyisocyanate. Examples of such polythiols include, but are not limited to, 1,4-benzene dithiol and 3,4-dimercaptotuloene.

### Polyisocyanate Component Description

Suitable organic polyisocyanates include any of those known in the art for the preparation of polyurethane foams, like aliphatic, cycloaliphatic, araliphatic and, preferably, aromatic polyisocyanates, such as toluene diisocyanate in the form of its 2,4 and 2,6-isomers and mixtures thereof and diphenylmethane diisocyanate in the form of its 2,4′-, 2,2′- and 4,4′-isomers and mixtures thereof.

An example of a suitable common polyisocyanate is an 80/20 isomer mixture of 2,4 and 2,6 toluene diisocyanate known as MONDUR TD-80, Grade A, which is commercially available from Bayer Corporation. Such toluene diisocyanates have a functionality of 2. Typically, the use of such toluene diisocyanates in preparing a flexible foam is associated with a TDI index. A TDI index is the value representing the amount of toluene diisocyanate (TDI) available for reaction with the polyol, water and other active-hydrogen sources in a foam producing formulation. An index of 105, for example, indicates that there is a 5%, excess of toluene diisocyanate equivalents available over the stoichiometric (i.e., the exactly proportional) amount required by the formulation.

Suitable diphenylmethane diisocyanates (MDI's) include mixtures of diphenylmethane diisocyanates and oligomers thereof having a polyisocyanate functionality greater than 2, known in the art as “crude” or polymeric MDI (polymethylene polyphenylene polyisocyanates), the known variants of MDI comprising urethane, allophanate, urea, biuret, carbodiimide, uretonimine and/or isocyanurate groups. The variants can be obtained by introducing uretonimine and/or carbodiimide groups in the polyisocyanates, such as uretonimine and/or carbodiimide modified polyisocyanate having an NCO value of at least 20% by weight, and/or by reacting such a polyisocyanate with one or more polyols having a hydroxyl functionality of 2-6 and a molecular weight of 62-500 to obtain a modified polyisocyanate having an NCO value of at least 20% by weight.

Mixtures of toluene diisocyanate and diphenylmethane diisocyanate and/or prepolymers thereof (adjusting the functionality number below accordingly) and/or polymethylene polyphenylene polyisocyanates also can be used. For example, polyisocyanates can be used which have an average polyisocyanate functionality of 1.7 to 3.5 and preferably 1.8 to 3.2.

### Foam Process Description—Molded

In general, a typical molded foam manufacturing facility for molding foams, in particular polyurethane foams, will consist of a metered foam mixing and dispensing unit, molds of a desired design, a mold conveying system, ovens, a roller crushing device, and related finished-foam handling systems. Such foam manufacture is typically automated where ever possible through the use of robotic devices.

The production of molded polyurethane foam typically involves the steps of mixing and dispensing a foam intermediate composition (i.e., combined “A” and “B” sides) as described herein from a foam dispensing unit, as a foam intermediate composition, into a sufficiently heated mold of a desired design. The mold is typically vented to allow for the build-up and subsequent release of internal pressure, has two or more sections with provisions for automatic opening and closing, and can be formed from cast aluminum or any other suitable material.

Following the mixing and dispensing steps, the lid of the mold is closed and locked, and the foam intermediate composition is allowed to cure at a sufficient temperature, for a sufficient period of time. A sufficiently heated oven capable of receiving the mold can also be employed during the curing step.

Once the curing step is completed, the lid of the mold is opened and the resultant flexible foam product is removed. This process step is typically referred to in the flexible foam manufacturing industry as demolding. After demolding, the resultant foam product is typically delivered along a conveyor system to a foam cell-crushing device. The crusher device is used to apply pressure to resultant flexible foam product to open the cells prior to being processed via other related finished-foam handling systems such as trimming and fabrication. During trimming and fabrication, the resultant flexible foam product is converted into a finished product such as an automobile seating cushion.

The viscosity determinations for the working examples are done in the conventional way using Brookfield rotational viscometers. For data at 25° C., samples in one quart, wide-mouth glass jars are measured using Brookfield's Model LVT viscometer.

The free-rise profile is an indication of how the foam will rise in the mold. This factor is important for two reasons. First, fill time is an important factor because the foam intermediate composition is poured into an open mold that typically is closed and locked before the rising foam fills the mold. If the closing lid touches the rising foam, gross foam instability problems will result. Second, fill time is also important for mold venting concerns. As the foam rises in the closed mold, the displaced air exits the mold through various vent holes. The timing of the closing for each vent device is critical so valuable foam material does not escape through the vent holes and become scrap.

### Foam Process Description—Slabstock

The production of a slabstock/one-shot foam is typically done utilizing either a higher pressure or low pressure machine containing continuous mixers. Such pressurized continuous mixing machines will generally produce 100 pounds or more per minutes of the slab stock foam to maintain an economic operation.

In general, slabstock foam production involves metering foam ingredients from separate feed lines (i.e., streams) via a mixing head containing a pin mixer or high shear mixer. Typical slabstock foam ingredients include a polyol such as glycerine-initiated propoxylate having an OHV of about 56; a polyisocyanate such as toluene diisocyanate; a diamine such as triethylene diamine-silicone; a blowing agent such as water, and a catalyst such as tin catalyst-alkyl morpholine and/or another polyol.

The temperature of the ingredients, commonly referred to as foam intermediates when mixed, is usually kept between 21° C. and 32° C. However, once a particular slabstock foam formulation is set, the temperature should be maintained within +/−1° C. since the density and other properties of the resultant foam are dependent upon the temperature.

Typically, slabstock foam density increases as the temperature of the foam intermediates exceeds 32° C. due to the result of temperature-dependent variations in the rates of competing isocyanate-hydroxyl and isocyanate-water reactions. Thus, if the foam intermediates are allowed to exceed a temperature of 32° C., gelation proceeds faster than the evolution of carbon dioxide resulting in a slabstock foam mass which resists further expansion and causes the formation of closed cells and spits (i.e., characteristics of increased foam density).

Once the foam ingredients are mixed, they are usually deposited from the mixing head in the form of a precise charge onto a sufficiently wrinkle-free release paper that is situated upon a conveyor belt system. The conveyor belt system is sufficiently angled away (i.e., 4-9°) from the mixing head while traversing (i.e., 5-10 ft./min) and conveying (i.e., 60-120 ft./min) at a sufficient speeds to maintain a uniform lay down of the foam.

After the foam ingredients are dispensed upon the release paper they are allowed to achieve a sufficient cream time and rise time. Cream time is defined as the time interval between the liquid ingredients leaving the mixing head and the commencement of the material turning white while on the conveyor. A typical cream time is between about 5-12 seconds. Rise time, on the other hand, is a measure of the time elapsed between the liquid leaving the mixing chamber and the completion of the expansion of the foam. Rise time is dependent upon the combined amine and catalyst concentrations in the foam formulation. A typical rise time is between about 80-160 seconds.

Following the foam mixture being allowed to sufficiently rise, it is usually conveyed to and placed under curing lamps, heaters or low pressure steam to eliminate surface tack to complete its processing into a final resultant foam product. Crushing or flexing of the resultant slabstock foam can also be done once the foam has finished the curing step. In doing so, more uniform loadbearing characteristics are achieved. Thus, most slabstock foams are generally crushed under a sufficient pressure for a sufficient amount of time as soon as they are tackfree (i.e., after about 15-30 minutes).

Once the slabstock foams are crushed, they can be conveyed to finished foam processing machinery to be cut and/or split into usable forms. The most common slabstock finished foam processes employed are centrifugal peeling and horizontal table splitting. Moreover, dielectric heat sealing or welding equipment can also be employed to seal pieces of slab foam either to itself or to a variety of plastic films. Compression devices can also be utilized to contour and shape the resultant slab foam. In doing so, decorative and field of application characteristics for the resultant foam can be achieved.

Further information regarding the production of slabstock foam can be observed in the following prior art references: Frish, K. C. and Saunders, J. H., Polyurethanes: Chemistry and Technology Part II, High Polymers, vol. XVI, part II, pp. 85-191 (1983) and Sandridge, R. L., et al., Effect of Catalyst Concentrations on One-shot Polyether Flexible Urethane Foams, American Chemical Society, Division of Organic Coating Plastics Chemistry, Preprints (1961), 21(no. 2), pp. 68-78. Such references are illustrative of prior art slabstock foam processing, but are not intended to be an exhaustive list.

### Material Handling

Another practical requirement of many molded foam producers is that preferably no individual foam ingredient should exceed a viscosity of about 10,000 mPa·s. The practical significance of this requirement relates to those producers' ability to off-load the product into their storage tanks and further handle the material within their respective production plants. Shipping the material at an elevated temperature is not usually an accepted option. The viscosity of embodiments of the present technology can be similar to that of conventional copolymer polyols such as HYPERLITE E-849. In a most preferred form, embodiments of the present technology do not exhibit a material-handling problem for producers.

### Load-Bearing Testing and Test Results

The ability of a flexible polyurethane foam to receive and support weight is referred to as its load-bearing capacity. This property is typically quantified by measuring Indentation Force Deflection (IFD) as specified in ASTM D3574.

To be a good cushioning material, a foam desirably exhibits both comfort and support properties. Comfort results when a material is able to deflect its surface under low loads and conform to body shape. For foams, the comfort characteristics are typically judged up to about 25% deflection. Beyond 25% deflection, a foam desirably exhibits support characteristics adequate to hold the human body in the desired position. Thus, to avoid bottoming out, the design firmness of a cushioning product is typically set at 65% deflection.

The ring-containing isocyanate reactive component molecules that are incorporated into the prepolymer of the present technology demonstrate an ability to build load-bearing in an actual molded foam. Although not wanting to be bound by any particular theory, it is believed that by varying the use level and the choice of polyether polyol in the formulation, the ring-containing component of the present technology could allow for the substantial reduction or replacement of copolymer polyols in foam applications such as seat cushioning. To do so would overcome the shortcomings of the conventional foam formulations described in the Background of the Invention section above, while also providing cost savings.

### Compressive Strength Testing

Compressive strength is a measurement of the ability of a foam to withstand a compressive force. A standard method for testing compressive properties is specified in ASTM D1621. Another method of measuring the compressive strength of a flexible foam makes use of an Instron® portable testing instrument. In this method, an Instron® testing instrument, model designation In-Spec 2200 Handheld Tester, is mounted on a rigid support and fitted with an indenter which has a radiused contact face, e.g., a sphere or section of a sphere. When a flexible foam is produced, a thin strip is cut off the outer edges of the foam and discarded. Immediately after this cut is made, the foam is removed from the production line, and the cut edge surface tested for compressive strength using the Instron® testing instrument. A jig can be used to hold the foam in place while the force is applied with the instrument. The foam can also be tested at any later time desired, using the same procedure.

### Compression Set Testing and Test Results

Another property of flexible polyurethane foams is compression set which is an indication of the ability of a flexible foam to retain its thickness after being exposed to constant deflection. This property is determined in accordance with ASTM D3574-03 “Standard Test Methods for Flexible Cellular Materials—Slab, Bonded and Molded Urethane Foams,” Test D “Constant Deflection Compression Set Test.” The test method consists of deflecting the foam specimen to a specified deflection, exposing it to the specified conditions of time and temperature, and then measuring the change in thickness of the specimen after a specified recovery period. In the examples set forth below, the compression set test results were determined by subjecting the foam specimens to deflections of 50% and 90% for a period of 22 hours at 158° F. (dry heat, i.e. substantially free of added moisture) and then measuring the thickness change of the foam specimens. Additional description of the compression set can be found in the book “Dow Polyurethane Flexible Foams” 2^{nd} Ed., R. Harrington, K. Hock, Editors, Chapter 7.

### Comparative Load-Bearing Efficiency Testing and Test Results

Foam manufacturers are interested in achieving a given load-bearing at a given or reduced foam density and at reduced raw material costs. Load-enhancing technologies are often compared in terms of how many additional Newtons of force are required to deflect the foam, per part of load-bearing additive in the foam formulation. Higher numbers indicate a higher efficiency for introducing load-bearing characteristics, which (at comparable raw material pricing) can translate into a less expensive foam pad. The load efficiency numbers are calculated for example by dividing the 65% IFD gain of the foam by the amount of load-bearing additive added. Such a calculation results in Newtons per part data. The total available range of Newtons is calculated as the practical use range times the efficiency number.

### WORKING EXAMPLES

### Prepolymer Preparation

A prepolymer in accordance with the present technology was prepared by reacting a polyester polyol derived from the reaction of phthalic anhydride with diethylene glycol (nominal OHV=135) with a toluene diisocyanate, specifically an 80/20 isomer mixture of 2,4 and 2,6 toluene diisocyanate known as MONDUR TD-80, available from Bayer Corporation. The prepolymer was prepared by adding 40% by weight of the polyester polyol to the diisocyanate. The mixture was stirred under a nitrogen blanket for approximately 2 hours at about 70° C. The resulting isocyanate terminated prepolymer had an NCO content of 24.7% and a viscosity (at 25° C.) of 903 cps. The NCO/OH ratio was approximately 7:1.

### Foam Preparation

The polyester polyol-containing prepolymer described above was mixed with additional (unreacted) MONDUR TD-80 toluene-diisocyanate to form an A-side blend comprising 30% prepolymer and 70% diisocyanate. The A-side blend is then mixed with a polyether resin blend (B-side) in the ratio of 38.7 (A-side) to 61.3 (B-side) in accordance with the Formulations 1 and 2 shown in Table 1, and prepared into slabstock flexible foams using water as the blowing agent. A control formulation, which contains no prepolymer, is also included for comparison purposes.

TABLE 1
Foam Formulations

| Component | Control | Formulation #1 | Formulation #2 |
|---|---|---|---|
| B-Side | | | |
| ARCOL F-3222 | 44.0 | 40.3 | 45.9 (% in Foam) |
| HS 100 (CPP) | 19.0 | 17.3 | 11.5 |
| Niax L-620 | 0.50 | 0.46 | 0.46 |
| T-Cat 110 | 0.14 | 0.13 | 0.13 |
| DABCO 33 LV | 0.09 | 0.09 | 0.09 |
| Niax A-1 | 0.06 | 0.06 | 0.06 |
| Water | 2.83 | 2.84 | 2.84 |
| A-Side | | | |
| Pure TDI | 33.38 | 27.10 | 27.30 |
| Prepolymer | 0 | 11.62 | 11.70 |

### Description of Materials and Abbreviations

In the formulations for the working examples, summarized in Table 1, the following materials were used:

ARCOL F-3222 is a propoxylated polyether triol with 12% internal ethylene oxide and a nominal OH value of 52.

CPP—an abbreviation for Copolymer Polyol. The solids content for each of the copolymer polyol containing foam runs was derived from HS-100, a copolymer polyol with a nominal 43 weight % styrene/acrylonitrile solids content and a nominal OH value of 28.

DABCO 33-LV—A commercial catalyst from Air Products Company consisting of 33 weight % triethylene diamine in dipropylene glycol.

NIAX L-620 is a silicone-based surfactant.

HS 100 is a copolymer polyol with a nominal 43 weight % styrene/acrylonitrile solids content and a nominal OH value of 28.

MONDUR TD-80 Grade A is an 80/20 isomer mixture of 2,4 and 2,6 toluene diisocyanate supplied by Bayer Corporation.

NIAX-A-1 is a catalyst supplied by Crompton-OSI Specialties consisting of 70 weight % bis(dimethylaminoethyl) ether in dipropylene glycol.

T-Cat 110 is a stannous octoate catalyst.

### Test Results

The essential physical properties of the foams produced from Formulations 1 and 2 were determined and compared to the properties of the control formulation. The results are set forth in Table 2. The indentation force deflection values at 25% and 65% deflection (IFD), , and the compression set, were measured according to ASTM D3574.

TABLE 2

| Physical Property | Control | Formulation #1 | Formulation #2 |
|---|---|---|---|
| Core density (kg/m³) | 21.8 | 21.8 | 21.1 |
| 25% IFD (N/323 cm²) | 173.5 | 204.6 | 169.0 |
| 65% IFD (N/323 cm²) | 302.5 | 369.2 | 293.6 |
| Tensile strength (psi) | 17.6 | 16.9 | 17.3 |
| Tear strength (psi) | 3.1 | 2.9 | 2.7 |
| Elongation % | 166 | 165 | 154 |
| 50% Compression Set, % | 6.3 | 8.5 | 8.2 |
| 90% Compression Set, % | 15.7 | 14.2 | 16.1 |
| Air Flow (cfm) | 4.3 | 3.6 | 3.9 |

From the results shown in Table 2, it can be seen that Formulation 1, comprising the ring-containing polyol in the prepolymer composition, provides enhanced load-bearing properties (i.e., increased values at 25% IFD and 65% IFD) compared to the control formulation with minimal loss of other physical properties. It can also be seen that Formulation 2, comprising the ring-containing polyol in the prepolymer composition, provides nearly equivalent load-bearing properties as the control, yet uses about 33% less copolymer polyol than the control. Further, Formulation 2 also shows a minimal loss of other physical properties, demonstrating that the ring-containing polyol prepolymer can be substituted for the copolymer polyol without sacrificing physical properties of the flexible foam.

In addition, compositions are contemplated that employ less or none of a dispersion of solids (a copolymer polyol) as the load-bearing contribution additives. Reducing or eliminating the solids is expected to reduce the variability of solids loading in the formulation, thus reducing the variability in load-bearing performance of the resulting foam.

While particular elements, embodiments and applications of the present invention have been shown and described, it will be understood that the invention is not limited thereto since modifications may be made by those skilled in the art, particularly in light of the foregoing teachings. Therefore, it is understood that the embodiments described above are merely for illustrative purposes and are not intended to limit the spirit and scope of the invention, which is defined by the following claims as interpreted according to the principles of patent law, including the doctrine of equivalents.

What is claimed is:

1. A prepolymer suitable for use in preparing a flexible foam comprising:
a. at least about 35% by weight of the prepolymer of a ring-containing isocyanate reactive component having load-bearing characteristics, the ring-containing isocyanate reactive component having an isocyanate reactive functionality between 1.7 and 3.5; and
b. no greater than about 65% by weight of the prepolymer of a polyisocyanate.

2. The prepolymer of claim 1, wherein the ring-containing isocyanate reactive component is selected from the group consisting of ring-containing polyols, ring-containing polyamines, ring-containing polyamides, ring-containing polyepoxides, ring-containing polycarboxylic acids, ring-containing polyanhydrides, and ring-containing polythiols.

3. A prepolymer suitable for use in preparing a flexible foam comprising:
a. at least about 35% by weight of the prepolymer of a non-halogenated aromatic polyester polyol or polyol blend having a hydroxyl functionality between 1.7 and 3.5, the non-halogenated aromatic polyester polyol being the reaction product of an aromatic dibasic acid, aromatic anhydride, aromatic diester or mixture thereof with a glycol or glycol mixture, the aromatic dibasic acid, aromatic anhydride, aromatic diester or mixture thereof comprising less than 20 mole percent aliphatic monobasic acid, aliphatic dibasic acid, aliphatic anhydride or aliphatic diester; the polyol being substantially free of aromatic diamines; and
b. no greater than about 65% by weight of the prepolymer of a polyisocyanate.

4. A prepolymer comprising:
a. at least about 35% by weight of the prepolymer of a non-halogenated ring-containing component polyol having a hydroxyl functionality between 1.7 and 3.5, the non-halogenated ring-containing polyol being the reaction product of a ring-containing component aliphatic dibasic acid, ring-containing component aliphatic anhydride, ring-containing component aliphatic diester or mixture thereof with a glycol or glycol mixture, the ring-containing component aliphatic dibasic acid, anhydride, ester or mixture thereof comprising less than 20 mole percent linear aliphatic monobasic acid or aliphatic dibasic acid; the polyol being substantially free of aromatic diamines; and
b. no greater than about 65% by weight of the prepolymer of a polyisocyanate.

5. A prepolymer comprising:
a. at least about 35% by weight of the prepolymer of a non-halogenated ring-containing component polyol having a hydroxyl functionality between 1.7 and 3.5; the non-halogenated ring-containing components polyol comprising a non-polyester polyol; the polyol being substantially free of aromatic diamines; and
b. no greater than about 65% by weight of the prepolymer of a polyisocyanate.

6. A prepolymer comprising:
a. at least about 35% by weight of the prepolymer of a halogen-containing, ring-containing component polyol or a halogen-containing, ring-containing component polyol blend having a hydroxyl functionality between 1.7 and 3.5, the halogen-containing, ring-containing component polyol being substantially free of any inorganic particulate solids; and
b. no greater than about 65% by weight of the prepolymer of a polyisocyanate.

7. The prepolymer of claim 3, wherein the non-halogenated polyester polyol consists essentially of the reaction product of phthalic anhydride and diethylene glycol.

8. The prepolymer of claim 1, wherein the prepolymer comprises at least about 40% by weight of the ring-containing isocyanate reactive component.

9. The prepolymer of claim 1, wherein the polyisocyanate comprises toluene diisocyanate.

10. The prepolymer of claim 3, wherein the prepolymer comprises at least about 40% by weight of the aromatic polyester polyol.

11. The prepolymer of claim 3, wherein the polyisocyanate comprises toluene diisocyanate.

12. The prepolymer of claim 4, wherein the prepolymer comprises at least about 40% by weight of the ring-containing component polyol.

13. The prepolymer of claim 4, wherein the polyisocyanate comprises toluene diisocyanate.

14. The prepolymer of claim 5, wherein the prepolymer comprises at least about 40% by weight of the non-polyester polyol.

15. The prepolymer of claim 5, wherein the polyisocyanate comprises toluene diisocyanate.

16. The prepolymer of claim 6, wherein the prepolymer comprises at least about 40% by weight of the halogen-containing, ring-containing polyol.

17. The prepolymer of claim 6, wherein the polyisocyanate comprises toluene diisocyanate.

18. A flexible foam prepared by reacting ingredients comprising:
a. a prepolymer comprising at least about 35% by weight of the prepolymer of a ring-containing load-bearing polyol and no greater than about 65% by weight of the prepolymer of a polyisocyanate; and
b. a polyether polyol;
c. in the presence of a surfactant, a blowing agent and a reaction catalyst; said foam having a density of from about 6 to about 240 kg/m² and a 65% indentation force deflection of from about 250 N/323cm² to about 500 N/323 cm².

19. The flexible foam of claim 18, wherein the blowing agent is water.

20. The flexible foam of claim 18, having a density of from about 8 to about 160 kg/m³.

21. The flexible foam of claim 18, having a density of from about 24 to about 64 kg/m³.

22. A foam intermediate composition suitable for use in making a flexible foam, the foam intermediate composition comprising:
a. a prepolymer comprising at least about 35% by weight of the prepolymer of a ring-containing load-bearing polyol and no greater than about 65% by weight of the prepolymer of a polyisocyanate;
b. a polyether polyol;
c. a surfactant;
d. a blowing agent; and
e. a reaction catalyst.

23. The foam intermediate composition of claim 22, wherein the composition further includes additional polyisocyanate mixed with the prepolymer.

24. The foam intermediate composition of claim 22, wherein the composition further includes at least one copolymer polyol in an amount of less than about 30% by weight of the foam intermediate composition.

25. The foam intermediate composition of claim 23, wherein the mixture of the prepolymer and the additional polyisocyanate comprises from about 30% to about 50% by weight of the foam intermediate composition.

26. A method of making a flexible foam comprising the steps of:
a. forming a prepolymer composition comprising at least about 35% by weight of the prepolymer of a ring-containing load-bearing polyol and no greater than about 65% by weight of the prepolymer of a polyisocyanate; and
b. reacting the prepolymer composition with a polyether polyol in the presence of a surfactant, a blowing agent and a reaction catalyst.

27. The method of claim 26, wherein the prepolymer is mixed with additional polyisocyanate prior to reacting the prepolymer with the polyether polyol.
